(19) United States
(12) Patent Application Publication
(10) Pub. No.: US 2011/0270362 A1
(43) Pub. Date: Nov. 3, 2011

(54) ACTIVE CIRCUIT MRI/EMI PROTECTION POWERED BY INTERFERING ENERGY FOR A MEDICAL STIMULATION LEAD AND DEVICE

(75) Inventors: Steven D. Goedeke, Forest Lake, MN (US); Gregory J. Haubrich, Champlin, MN (US); John D. Wahlstrand, Shoreview, MN (US)

(73) Assignee: Medtronic, Inc.

(21) Appl. No.: 12/884,367

(22) Filed: Sep. 17, 2010

Related U.S. Application Data
(60) Provisional application No. 61/328,841, filed on Apr. 28, 2010.

(51) Int. Cl. A61N 1/05; A61N 1/08

(52) U.S. Cl. 607/63; 607/116

(57) ABSTRACT

An implantable lead for use with a medical device (IMD) includes active circuits incorporated into the lead to reduce the creation of an induced current, or dissipate the induced current and heat created due to an induced current in the lead. The active circuits are powered by the magnetic resonant imaging energy or interfering magnetic or electrical fields. According to various embodiments, the lead and/or its components can be provided to reduce or dissipate a current and heat induced by various external magnetic or electrical fields.

### RELATED APPLICATION

This application claims priority from U.S. Provisional Application No. 61/328,841, filed Apr. 28, 2010, the content of which is incorporated herein by reference in its entirety.

### TECHNICAL FIELD

The present disclosure relates to implantable medical devices (IMDs), and in particular to a system and method for use of an implanted lead that includes active magnetic resonance imaging/electromagnetic interference (MRI/EMI) protection powered by and responsive to an interfering external field.

### BACKGROUND

The statements in this section merely provide background information related to the present disclosure and may not constitute prior art.

The human anatomy includes many types of tissue that can either voluntarily or involuntarily, perform certain functions. However, after disease or injury, certain tissues may no longer operate within general anatomical norms. For example, after disease, injury, age, or combinations thereof, the heart muscle may begin to experience certain failures or deficiencies. Some of these failures or deficiencies can be corrected or treated with implantable medical devices (IMDs). These devices can include implantable pulse generator (IPG) devices, pacemakers, implantable cardioverter-defibrillator (ICD) devices, cardiac resynchronization therapy defibrillator devices, or combinations thereof

One of the main portions of the IMD can include a lead that is directly connected to tissue to be affected by the IMD. The lead can include a distal end that is located adjacent to the tissue, such as a muscle bundle brain tissue, cardiac tissue of a heart or the like, and a lead body that connects to the device body or therapeutic driving device. It is generally known that the device body or case portion can be implanted in a selected portion of the anatomical structure, such as in a chest or abdominal wall, and the lead can be inserted so that one or more electrodes of the lead are positioned at selected positions near or in the muscle or tissue to which therapy is provided. Additionally, the lead one or more electrodes can be positioned near nerves, such as spinal nerves, to provide therapy to a portion of a spinal column or other nerve within the patient.

The IMD generally remains with the patient during the rest of the patient's natural life. To that end, the IMD can be exposed to various environmental factors. For example, the patient may undergo a magnetic resonance imaging (MRI) procedure or other high frequency imaging procedures. In this case, portions of the IMD may act as an antenna and have current and thermal energy induced therein due to the MRI procedure. This induced heat can damage anatomical tissue thereby reducing the efficacy of therapy or sensing. Accordingly, reduction or dissipation of the induced current or thermal energy may be useful in certain circumstances.

### SUMMARY

An implantable medical device (IMD) can include implantable pulse generator (IPG) devices, implantable cardioverter-defibrillators (ICD), cardiac resynchronization therapy defibrillator devices, neurostimulators, spinal stimulators, drug pumps, or other implantable device or combinations thereof. The IMD can be positioned in a selected portion of the anatomical structure, such as a chest wall or abdominal wall, and a lead can be positioned so that a lead tip can be implanted in anatomical tissue. Various portions of the IMD, such as a case or device body, the lead body, or the lead tip, can be formed or augmented to reduce or dissipate heat production due to various external environmental factors. For example, a magnetic and/or electric field from a magnetic resonance imager (MRI), diathermy (including shortwave, microwave, or the like) or other energy field producing devices can induce currents in the lead. According to various embodiments, self-powered active circuits can be incorporated into the lead to reduce the creation of an induced current, or dissipate thermal energy created due to an induced current in the lead, to reduce or eliminate negative effects of the induced signal. The active circuits are self-powered by the incident interfering energy and thus uses the interfering field to combat any negative effects of the interfering energy. As used herein, interfering energy, interfering field, and interfering signal refer to an impinging energy, field or signal that may have a negative effect on operation of one or more components of the medical system.

An implantable medical device operable to provide therapy to an anatomical tissue is provided. An implantable medical device can include a housing having a controller including circuitry for generating the therapy and a lead having a proximal end coupled to the housing and a distal end located adjacent to an anatomical tissue of a patient. An electrode is positioned near the distal end of the lead and a conductor extends from the proximal end of the lead to the distal end of the lead such that the circuitry for generating therapy is in electrical communication with the electrode. At least one active circuit satellite is positioned within the lead and near the electrode and includes a power circuit for converting an interfering electromagnetic field into power for controlling one or more active circuits within the at least one active circuit satellite. When powered, each active circuit including circuitry performs an action to reduce negative effects of the interfering electromagnetic field. In other words, the active circuits become self-powered in the presence of an interfering external field and can take corrective action either immediately or upon confirming detection of the presence of a particular interfering field of interest.

In addition, an implantable lead is also provided that carries therapy to anatomical tissue. The lead includes a lead body having a proximal end configured to couple to a housing of the implantable medical device and a distal end configured to be located adjacent to an anatomical tissue of a patient. An electrode positioned near the distal end of the lead and a conductor extends from the proximal end of the lead to the distal end of the lead providing electrical communication along the lead to the electrode. One or more active circuits positioned within the at least one lead perform an action to reduce effects of an interfering electromagnetic field when powered by a power circuit positioned within the lead that converts the interfering electromagnetic field into power for the one or more active circuits such that the one or more active circuits become operational when the interfering electromagnetic field is present.

Further provided is a method of controlling a lead having active circuit satellites implanted in an anatomical structure during the presence of an interfering external field. The method can include converting an interfering electromagnetic field to a power signal and powering at least one active circuit positioned near a distal end of an implantable medical lead with the power signal. When powered the at least one active circuit and performs an action to reduce negative effects of the interfering electromagnetic field. The method can also include discontinuing corrective actions and resuming normal delivery of therapy when the external field is no longer present.

Further areas of applicability will become apparent from the description provided herein. It should be understood that the cardiac description and specific examples are intended for purposes of illustration only and are not intended to limit the scope of the present disclosure from applications in neuro stimulation, spinal stimulation or stimulation in any other part of a patient's body.

### BRIEF DESCRIPTION OF THE DRAWINGS

The drawings described herein are for illustration purposes only and are not intended to limit the scope of the present disclosure in any way.

FIG. 1 is a view of an IMD including a lead connected to a device body;

FIG. 2 is a simplified environmental view of the IMD implanted in a patient;

FIG. 3 is a simplified environmental view of an IPG that includes an exemplary cardiac lead system with a switch responsive to an external field, such as that generated during a magnetic resonance imaging (MRI) procedure;

FIG. 4 is a simplified perspective view of a ring electrode and the switch of the cardiac lead system of FIG. 3;

FIG. 5 is a simplified block diagram of an active circuit satellite;

FIG. 6 is another embodiment of a simplified block diagram of an active circuit satellite;

FIG. 7a-FIG. 7f are exemplary circuit diagram of the series active circuits of FIG. 5 and FIG. 6;

FIG. 8a-FIG. 8e are exemplary circuit diagram of the shunt active circuits of FIG. 5 and FIG. 6;

FIG. 9a-FIG. 9b are exemplary operational actions that may be taken by the active circuit satellites of FIG. 5 and FIG. 6;

FIG. 9c are exemplary operation actions for step 908 of FIG. 9a;

FIG. 10 are exemplary operational actions that may be taken by the implantable medical device or the active circuit satellites;

FIG. 11 is an illustration of an exemplary multiple active circuit satellite embodiment;

FIG. 12 is an illustration of an exemplary additional embodiment for the implantable medical lead to protect the implantable medical device from MRI/EMI interference; and

FIG. 13 is an illustration of another embodiment using one electrode and the housing of the implantable medical device to create power from the MRI/EMI interference.

### DETAILED DESCRIPTION

The following description is merely exemplary in nature and is not intended to limit the present disclosure, application, or uses. It should be understood that throughout the drawings, corresponding reference numerals indicate like or corresponding parts and features. The present disclosure describes the techniques with reference to a system and method for use of a cardiac lead that includes a power circuit that converts an interfering electromagnetic field, such as that generated by an MRI, into a potential for powering one or more active circuits to become operational when the interfering electromagnetic field is present. The one or more active circuits may operate to reduce or eliminate negative effects of the interfering electromagnetic field on the patient or on circuitry within the implantable medical device. It should be noted, however, that the present teachings could be applicable to other contexts (e.g., neuro stimulation or spinal stimulation) in which it is desirable to have a component that is responsive to interfering external fields. Additionally, the techniques may be used to reduce the effects of interfering external signals from devices other than MRI devices, such as diathermy devices (including shortwave, microwave, or the like), ablation devices, electrocautery devices, radiofrequency identification (RFID) security gates or other medical or non-medical source of interfering external signals.

Further, as used herein, the term circuit is intended to include discrete circuits, integrated circuits, a processor (shared, dedicated, or group) and memory that executes one or more software or firmware programs or instructions, combinational logic circuits, and/or other suitable software, firmware programs or components that provide the described functionality. As such, the disclosure further contemplates a computer-readable medium that contains instructions that, when executed by the processor or circuit, causes the device or one or more components of the device to perform the function(s) attributed to them. Therefore, it will be understood that the following discussions are not intended to limit the scope of the appended claims.

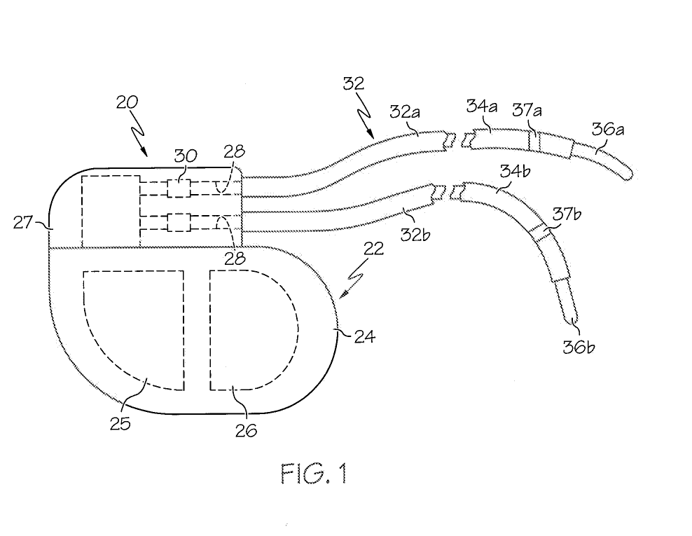

With reference to FIG. 1, an implantable medical device (IMD) 20, which can include implantable pulse generator (IPG) devices, implantable cardioverter-defibrillator (ICD) devices, cardiac resynchronization therapy defibrillator devices, or combinations thereof, is exemplarily illustrated. The IMD 20 can include an implantable case or body assembly 22. The implantable case 22 can be formed of appropriate materials and include appropriate features, such as a hermetically sealed body wall 24. The body wall 24 can be made of a substantially inert material or of a conducting material.

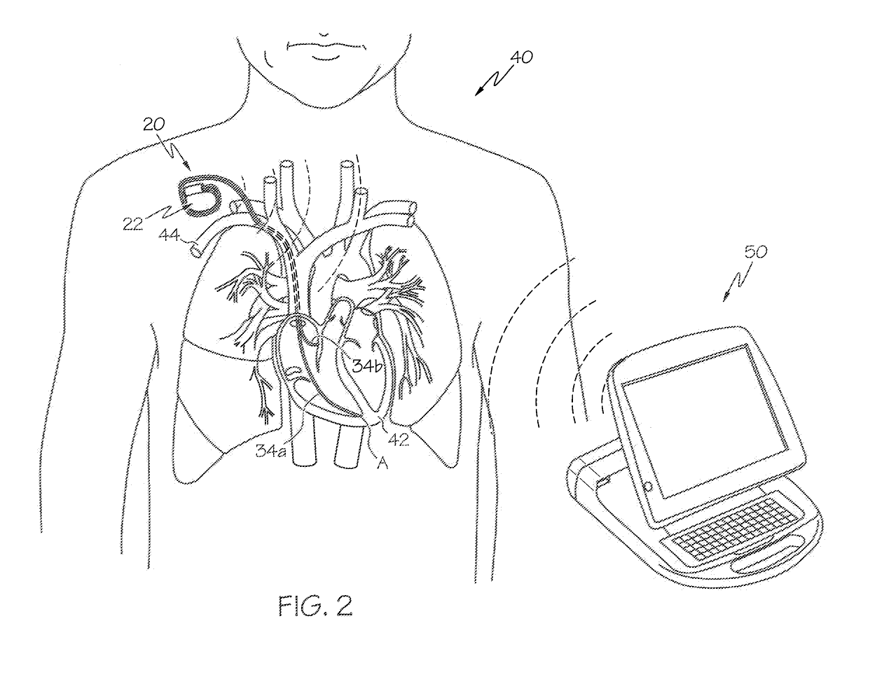

Contained within or associated with the case 22 can be a power device 25 (such as a battery), a controller assembly 26, and a connector body 27 (sometimes referred to as a header). The controller assembly 26 can include a circuit board having a processor, memory, transmitter, receiver, and other appropriation portions, further discussed herein. The connector body 27 can extend from or be integrated with the case 22. The connector body 27 can include multiple ports 28 that each interconnect with a respective connector terminal 30 of a lead assembly 32. FIG. 1 illustrates two lead assemblies 32a, 32b where each lead assembly 32a, 32b includes lead bodies 34a, 34b, respectively, extending from the connector body 27. Lead assemblies 32a, 32b include one or more electrodes located along a length of lead bodies 34a, 34b. In the example illustrated in FIG. 1, lead assemblies 32a, 32b include tip electrodes 36a, 36b and ring electrodes 37a, 37b located near a distal end of lead bodies 34a, 34b. Although the IMD 20 is illustrated in FIGS. 1 and 2 as including two lead assemblies 32a, 32b, it will be understood that any number of lead assemblies 32 could be employed with the IMD 20 depending upon the malady of the patient and the particular IMD 20 employed. Moreover, each of lead assemblies 32 may include more or fewer electrodes and/or different types of electrodes depending upon the malady of the patient or IMD 20 employed. For example, in some embodiments, the lead may include a plurality of ring electrodes 37 in addition to or instead of a tip electrode 36. The techniques described in this disclosure may be utilized within one or more of the lead assemblies 32a, 32b for the particular IMD 20 employed.

A fixation mechanism can also be included with each lead assembly 32a, 32b to affix each tip electrode 36a, 36b relative to or in a selected tissue of the patient. The fixation mechanism can be near each tip electrode 36a, 36b or define a portion of the tip electrode 36a, 36b. Fixation mechanisms (not shown in FIGS. 1 and 2) can be any appropriate type, including a grapple mechanism, a helical mechanism, a drug-coated connection mechanism, and other appropriate connection mechanisms.

Lead assemblies 32a, 32b also include one or more conductors (not shown in FIG. 1) that extend from a proximal end of lead bodies 34a, 34b to the distal end of lead bodies 34a, 34b. For example, a first electrical conductor can extend from each connector terminal 30 to engage each tip electrode 36a, 36b and a second electrical conductor can extend from each connector terminal 30 to engage each ring electrode 37a, 37b. In this example, each of lead assemblies 32a, 32b includes two conductors. The electrical conductors transmit the therapy from a therapy circuit (e.g., pulse generator) within IMD 20 to the respective one of the electrodes. It will be understood by one skilled in the art that the electrical conductors can each be separate pieces or multiple components that are interconnected (e.g., a multifilar wire). The electrical conductors can also be cannulated or include a solid or non-cannulated cable. A majority of each lead body 34a, 34b can also be formed in a generally known and selected manner. For example, various conductors and electrical components can be encased in silicone, polyurethane, and other appropriate materials. The casing material of each lead body 34a, 34b can electrically insulate the inner electrical conductor from an external environment.

The IMD 20, including the components discussed above, can be implanted in a patient 40 as illustrated in FIG. 2. The IMD 20 can include one or more lead assemblies 32, such as the first lead assembly 32a and the second lead assembly 32b. The first lead assembly 32a and the second lead assembly 32b can be connected to the connector body 27. As one skilled in the art will understand, the position of lead bodies 34a, 34b can depend upon the type of IMD and the malady of the patient 40. For example, the lead assemblies 32a, 32b can be positioned transvenously to positions within a heart 42 or on the outside of the heart 42. The IMD 20 can be provided to pace the heart 42, defibrillate the heart 42, cardiovert the heart 42, resynchronize the heart 42, sense conditions of the heart 42, or the like. In other applications, the lead may be positioned to simulate the brain, nerves, spine or other nerve, tissue, muscle or organ.

The IMD 20, including the case 22 and the lead bodies 34a, 34b, can be implanted using known procedures. For example, an incision can be made in a chest wall or an abdomen wall of the patient 40 and the lead assemblies 32a, 32b can be passed through selected veins to selected portions of the heart 42 of the patient 40. The case 22 can also be positioned through the incision into a chest wall or abdominal wall of the patient 40. In a selected procedure, the leads assemblies 32a, 32b can be passed through a superior vena cava 44 of the patient 40. The lead tips or tip electrodes 36a, 36b and ring electrodes 37a, 37b can be positioned at various positions in the heart 42, such as at the ventricles or atriums thereof. The position of the lead assemblies 32a, 32b, tip electrodes 36a, 36b and ring electrodes 37a, 37b can be selected for pacing, defibrillation, sensing, or other appropriate procedures. The specific implantation procedure, position of the tip electrodes 36a, 36b, ring electrodes 37a, 37b, and the like can depend upon the patient 40, the surgeon performing the procedure, the specifics of the lead assemblies 32a, 32b, or other considerations.

As discussed above, the IMD 20, including the case 22 and the lead assemblies 32a, 32b can include various features or controls to defibrillate, pace, cardiovert, or resynchronize the heart 42. The controls can include a processor associated with the controller assembly 26 located within the case 22. The processor can be programmed to control driving of a current through the lead bodies 34a, 34b to various combinations of the tip electrodes 36a, 36b and ring electrodes 37a, 37b to defibrillate, pace, cardiovert, or resynchronize the heart 42.

With continued reference to FIG. 2, a programmer or programming system 50 can be provided. The programmer 50 can include a telemetry system that is operable to wirelessly transmit a signal to the controller within the case 22, e.g., via radio frequency (RF) signals. The controller includes communication circuitry for bi-directional communication with the programmer 50. It will be understood that a wired communication system can also be used. In addition, an induction system can be used where a coil is positioned near the case 22 and a signal is sent from the programmer 50 via induction. The programmer 50 can also receive information from the IMD 20 (e.g. tachycardia rhythms, times and programming settings) to assist in providing an appropriate program for therapy and to determine if the IMD 20 is operating properly. The programmer 50 can include any appropriate programming system, including one generally known to those skilled in the art, such as the Medtronic CARELINK™ programmer, sold by Medtronic, Inc. of Minneapolis, Minn.

Moreover, the IMD 20, including the case 22 and the lead assemblies 32a, 32b, can be formed to counteract or interact with various environmental factors. For example, the lead assemblies 32a, 32b can include features or portions to re-direct or dissipate thermal energy created by an induced current. Induced currents can be created due to an external field, such as an electromagnetic field acting on the conductors of the lead assemblies 32a, 32b.

For example, according to various embodiments, the patient 40 which has the implanted IMD 20 may receive a certain therapy or diagnostic technique, such as an MRI scan. Although not illustrated, an MRI, generally understood by one skilled in the art, uses high frequency radio frequency (RF) pulses, a static magnetic field and gradient magnetic fields to create image data regarding the patient 40. An MRI may have a frequency of about 42 MHz per tesla. One common MRI system uses about 1.5 tesla magnetic fields and has a corresponding RF frequency of about 64 MHz. The techniques of this disclosure are not limited to such MRI systems. The techniques of this disclosure may be utilized with other MRI systems, such as a 3.0 tesla MRI system that has a corresponding RF frequency of about 128 MHz or other MRI system. Without being bound by the theory, the strong magnetic fields in a MRI can induce aligned spins of sub-atomic particles and the high frequency RF pulses can be used to change the alignment or otherwise affect the sub-atomic particles within the patient 40.

The gradient magnetic fields and RF pulses may induce currents within the lead assemblies 32a, 32b of the IMD 20. The current induced in the lead assemblies 32a, 32b may cause certain affects, including heating, of the various lead components or tissue of the patient 40 undergoing the MRI scan. Additionally, currents flowing toward the device 20 may affect some of the circuitry within the device. According to various embodiments, such as those discussed herein, components, controls and/or mechanisms can be provided to reduce or eliminate the amount of disruption caused by the interfering electromagnetic signals of the MRI scan, including the current that may affect the device or generate thermal energy at tip electrodes 36a, 36b or ring electrodes 37a, 37b.

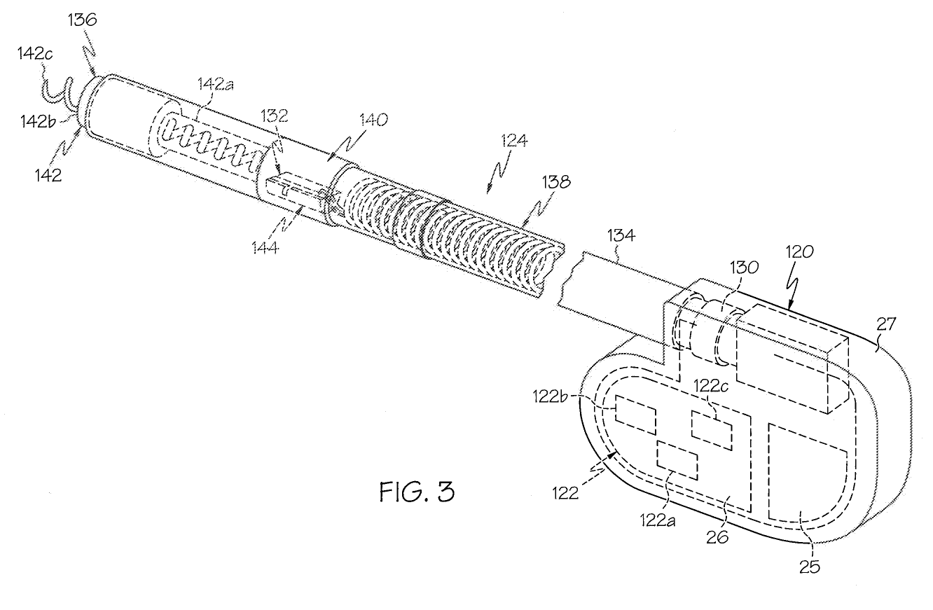

According to various embodiments, and with reference to FIG. 3, in one example the IMD 20 can comprise an implantable cardiac device, such as an implantable pulse generator (IPG) 120. In other applications, IMD 20 could comprise a neurostimulator, spinal stimulator or other implantable medical device. It should be noted that, while the IPG 120 is illustrated herein as including one lead assembly 124, the IPG 120 can include any number of lead assemblies (e.g., a uni-polar, bipolar or multi-polar lead) depending upon the malady of the patient 40. In one example, the IPG 120 can be used to generate electronic pulses and deliver the pulses to a desired location within the heart 42. In addition, the IPG 120 can sense electrical signals from the heart 42 to enable the IPG 120 to monitor the heart rhythm to determine if a therapy is needed.

Although described in the context of IPG 120, the techniques of this disclosure may be used in other contexts including other cardiac therapy and/or monitoring devices. Additionally, the techniques may be utilized within any device that provides electrical stimulation to a tissue site of a patient proximate a muscle, organ or nerve, such as a tissue proximate a vagus nerve, spinal cord, brain, stomach, pelvic floor or the like to treat various conditions, including movement and affective disorders such as chronic pain, Parkinson's disease, tremor and dystonia, urinary storage and voiding dysfunction, digestion dysfunction, sexual dysfunction or the like.

The IPG 120 can include a control system 122 and at least one IPG stimulation lead 124 which can be implanted into an anatomical structure, similar to the placement of the IMD 20 relative to the heart 42, as shown in FIG. 2. The control system 122 and the at least one IPG stimulation lead 124 can cooperate to reduce or eliminate the adverse effects caused by the interfering electromagnetic signals of the MRI scan, including the current that may damage the device or generate thermal energy at the tip electrode or ring electrode, as will be discussed. The control system 122 can include a controller 122a, a pulse generator 122b and a satellite communication circuit 122c that form specific parts of the controller assembly 26, illustrated in the IMD 20 discussed with regard to FIG. 1 and FIG. 2.

The controller 122a can be in communication with and responsive to the programmer 50 to receive a desired treatment plan for the heart 42, such as a desired voltage for the electrical stimulation of the heart 42. The controller 122a can also be in communication with the lead 124 to receive the sensed electrical activity of the heart 42 and transmit the sensed electrical activity to programmer 50, as will be discussed. As such, controller 122a may include a transmitter and/or receiver for wireless communication, e.g., via RF, inductance or the like. The pulse generator 122b can be in communication with and responsive to the controller 122a to generate the desired therapy (i.e., electrical stimulation or pulse) for the heart 42. The pulse generator 122b can be in electrical communication with the lead 124 to supply the lead 124 with the desired therapy, e.g., via at least one conductor.

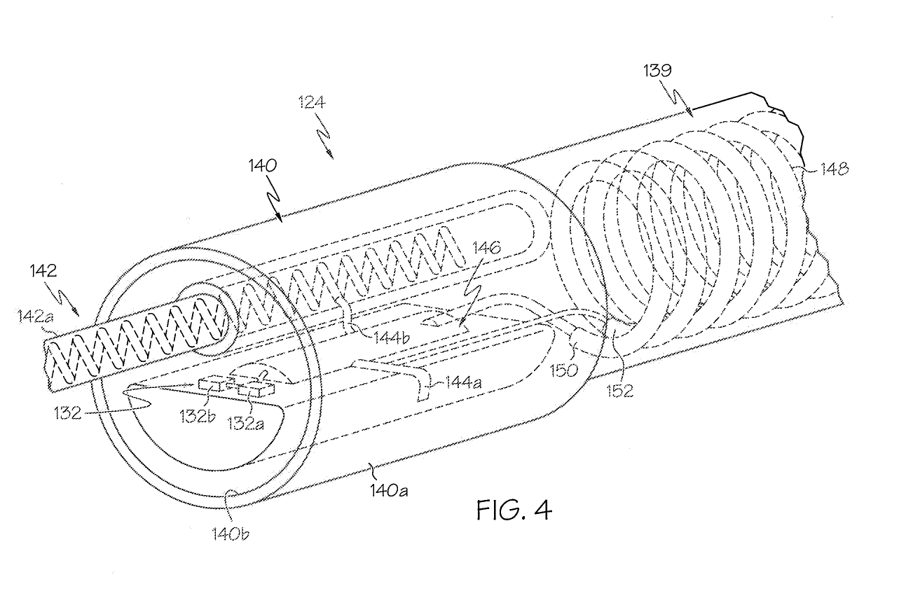

The satellite communication circuit 122c communicates with one or more active circuit satellites 146 (see, e.g., FIG. 4) positioned along the lead 124. The active circuit satellites 146 include at least one power circuit positioned within the lead 124 that converts an interfering electromagnetic field into a potential (e.g., direct current or alternating current) for powering one or more active circuits such that the one or more active circuits become operational when the interfering electromagnetic field is present. The one or more active circuits within the lead 124 each include circuitry for reducing or eliminating negative effects of the interfering electromagnetic field on the patient or on the circuitry for generating the therapy. As such, the active circuit satellites 146 are self-powered by the MRI/EMI signal, and in this way, the present disclosure describes techniques for using the interfering and potentially damaging energy to eliminate or reduce negative effects from such energy. Such negative effects include heating of patient tissue and/or damage to circuitry within the device.

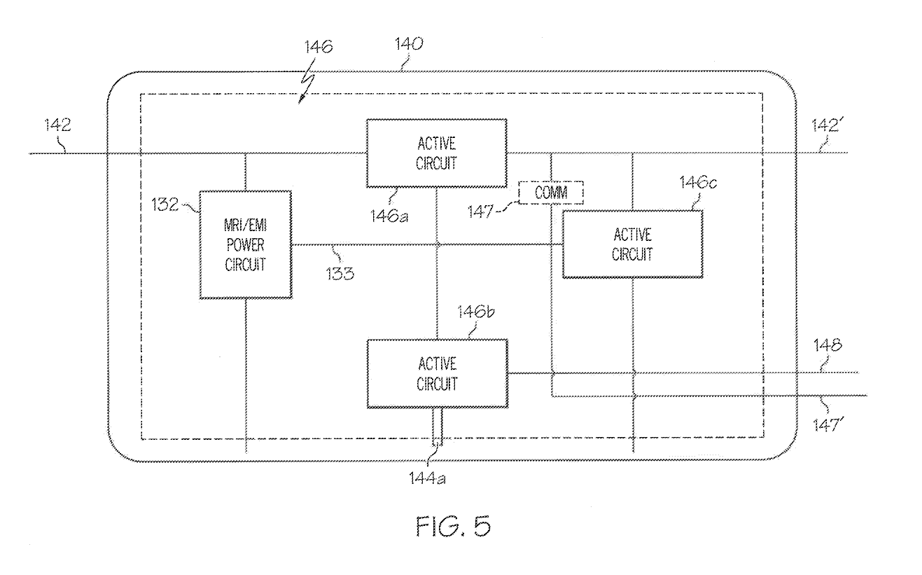

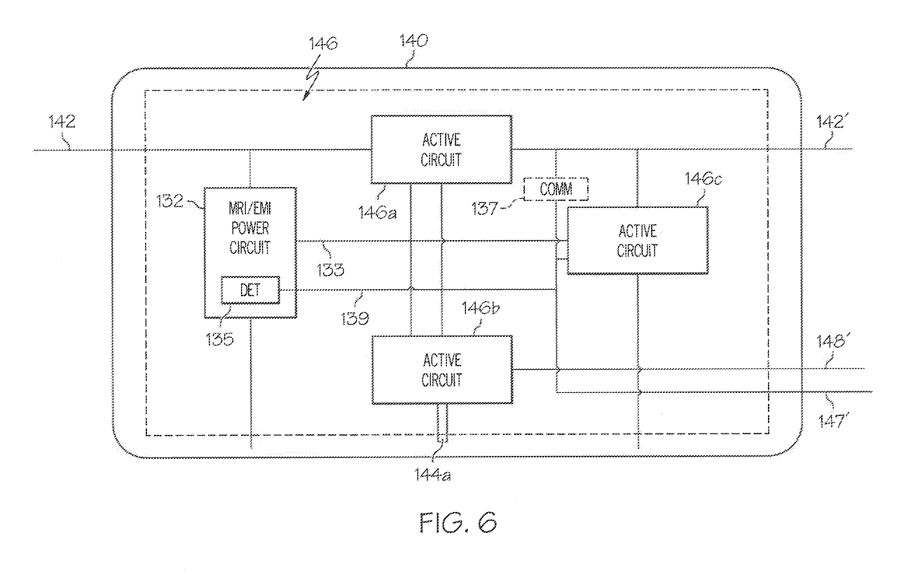

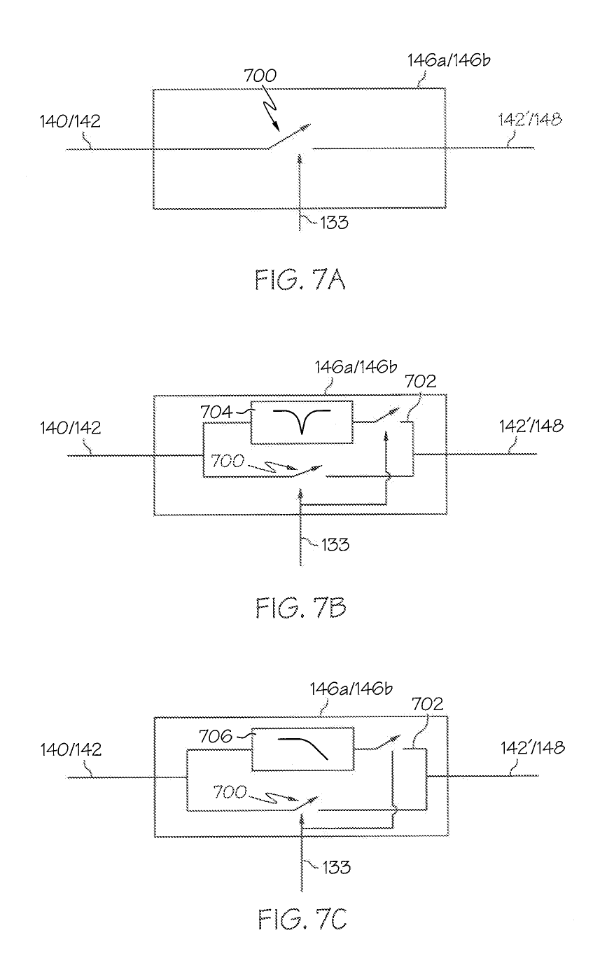

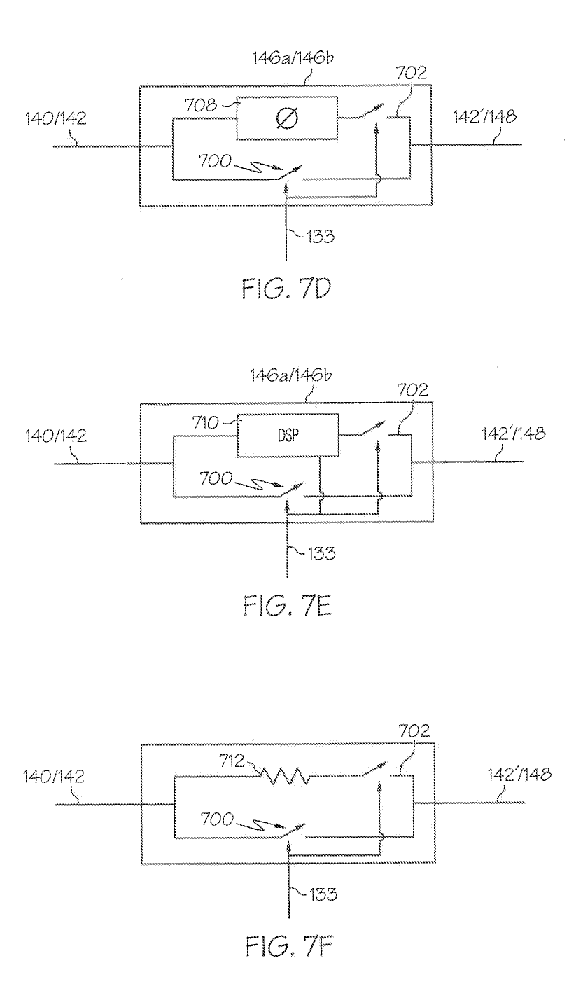

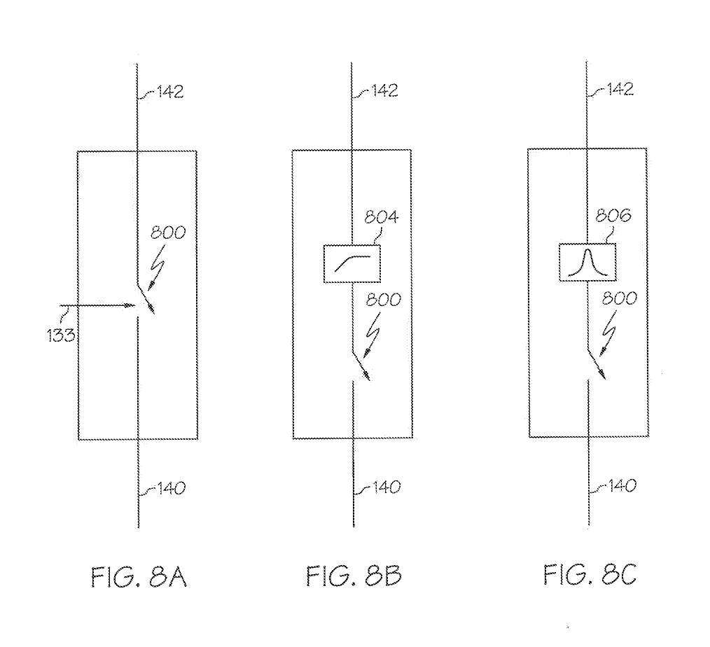

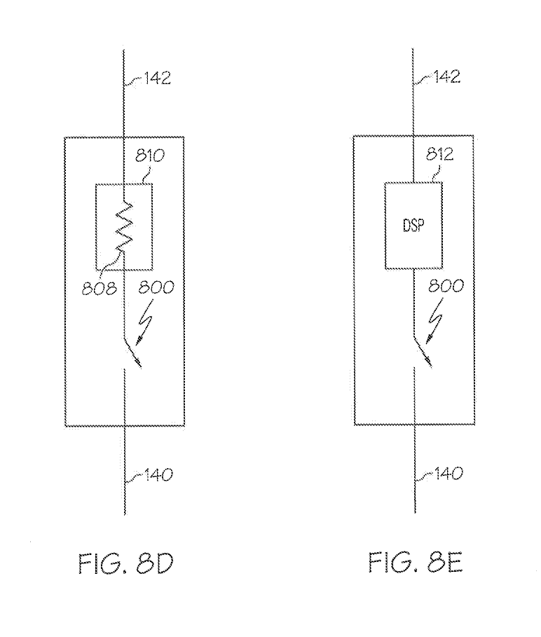

The active circuit satellite 146 may, in some instances, communicate with the satellite communication circuit 122c of IPG 120. In one embodiment, the active circuit satellite 146 may communicate with the satellite communication circuit 122c using a conductor via which therapy is provided from controller 122a to tip electrode 142 or ring electrode 140. This communication can be accomplished by any multiplexed communication scheme. For example, time division multiple access (TDMA) communication signals may be multiplexed with pacing pulses. In other examples other communication techniques may be used such as frequency division multiple access (FDMA), code division multiple access (CDMA), or any other channel access technique In another embodiment, multiplexed communication need not be used provided that the communication signals have an amplitude that are sub-threshold to a pacing pulse. Using the pacing conductor (e.g., the conductor that electrically couples either tip electrode 142 or ring electrode 140 to the pulse generator) has the advantage of reducing the size of the lead 124, which allows for a more flexible lead that may be easier for the physician to place. Alternatively, as discussed below in conjunction with FIG. 5 and FIG. 6, a separate conductor may be used for communication signals from the active satellite circuit 146 to the satellite communication circuit 122c of IPG 120 so that higher amplitude (and thus more immune to noise or MRI/EMI interference) communication signals can be used.

The communication between the satellite communication circuit 122c and the active circuit satellites 146 can be uni-directional or bi-directional. That is, in one embodiment, the active circuit satellite 146 communicates uni-directionally with the satellite communication circuit 122c. For example, active circuit satellite 146 may only send signals to the satellite communication circuit 122c. In this manner, the active circuit satellite 446 may alert the control circuitry 26 of IPG 120 that it is now operating in an adverse MRI/EMI field. In this way, therapy changes or other corrective actions can be taken by control system 122 of IPG 120 during the time the IPG 120 and lead 124 are within the interfering field.

In another embodiment, communication between the active circuit satellite 146 and the satellite communication circuit 122c is bi-directional or two-way. In this case, the satellite communication circuit 122c may send instructions to the active circuit satellite 146 regarding what actions to take with the now powered active circuits in addition to receiving alerts from active circuit satellite 146. Changes in actions taken can be based upon the strength or nature of the interfering field or can be programmed by the physician using the external programmer 50 discussed above in conjunction with FIG. 2.

With reference to FIG. 4, the active circuit satellite 146 is powered by a power circuit 132 that converts the interfering electromagnetic field into a potential for powering the one or more active circuits. In one example, power circuit 132 rectifies incident MRI/EMI energy into a direct current potential to power the active circuits in the active circuit satellite 146. Alternatively, power circuit 132 may convert the incident MRI/EMI energy into an alternating current for powering the active circuits. In one embodiment, the power circuit 132 is designed to require high energy fields, such as those generated during an MRI procedure, to sufficiently power the active circuits of the active circuit satellite 146. As described in more detail below, the active circuits operate to combat the incident energy upon being activated.

In another embodiment, the power circuit 132 is divided into a broad-band power circuit 132a such that any sufficiently strong energy field powers the active circuits and a narrow-band field sensor 132b to verify that a particular interfering energy field is present before allowing the active circuits to take an action to prevent the intrusion of the interfering energy field. In this case, active circuits do not take action until narrow-band field sensor 122b confirms that the field is a particular interfering energy field, e.g., an MRI-generated field. As such, the active circuits may take the action when in the presence of an MRI field (as detected by the narrow-band field sensor 132b) and not take the action when in the presence of another interfering field, e.g., a RFID security gate. A Hall effect sensor or the magnetic field sensor 60 disclosed in commonly assigned U.S. Pat. No. 7,050,855, incorporated herein by reference in its entirety can be used as the narrow-band field sensor 132b to verify the presence of a particular interfering electromagnetic field. Alternately, the field sensor 132b can comprise another suitable field sensor used to detect an external field.

Active circuit satellite 146 and/or power circuit 132 may be coated with a protective coating that is biocompatible with a patient. In one embodiment, active circuit satellite 146 and/or power circuit 132 is coated with a CHIPSKIN™ coating provided by Proteus Biomedical, Inc. of Redwood City, Calif. However, other biocompatible coatings may also be used. In another embodiment, active circuit satellite 146 and/or power circuit 132 may be enclosed within a hermetic enclosure. In one example, the hermetic enclosure may be sized to fit within ring electrode 140. In another example, the hermetic enclosure may be an in-line hermetic enclosure. An example in-line hermetic electronic package is described in U.S. Pat. No. 7,236,834 (referred to herein as “the '834 patent”) to Christopherson et al. entitled, “ELECTRICAL LEAD BODY INCLUDING AN IN-LINE HERMETIC ELECTRONIC PACKAGE AND IMPLANTABLE MEDICAL DEVICE USING THE SAME,” which is incorporated herein by reference in its entirety. As described in the ‘834 patent, the hermetic enclosure may located along the lead body such that a first portion of the lead body extending from the IMD is electrically coupled to one side of the hermetic enclosure and a second side of the hermetic enclosure is connected to a second portion of the lead body. In this manner, the hermetic enclosure that houses active circuit satellite 146 and/or power circuit 132 is in-line or in series with the lead body. In a further example, lead 124 may be constructed of a number of modular components that are assembled to form lead 124 and one of the module components may include active circuit satellite 145. An example lead constructed of a number of modular components is described in U.S. Pat. No. 6,473,653 to Schallhorn et al. entitled, “SELECTIVE ACTIVATION OF ELECTRODES WITHIN AN IMPLANTABLE MEDICAL DEVICE,” which is incorporated herein for its description of modular components and constructing a lead from modular components.

With reference to FIG. 5, the active circuit satellite 146 is shown in one embodiment to be positioned within the ring electrode 140. Alternately, the active circuit satellite 146 could be positioned anywhere along the lead as desired by a lead designer. A plurality of active circuits 146a, 146b and 146c are shown for an exemplary configuration. Those skilled in the art will appreciate that any number of active circuits may be employed depending upon the desired result of the lead designer. Also, the configuration of series and shunt active circuits may be modified by a lead designer to achieve a desired level of MRI/EMI protection. The two series and one shunt active circuits of FIG. 5 (and FIG. 6) are merely exemplary for ease of explanation of techniques of the present disclosure.

As can be seen in FIG. 5, active circuit 146a is coupled in series to the tip electrode 142, while active circuit 146b is coupled in series to the ring electrode 140 via connector 144a. A third active circuit 146c is coupled in a shunt configuration between the tip electrode 142 and the ring electrode 140. An MRI/EMI power circuit 132 is coupled between the tip electrode 142 and the ring electrode 140 to process any interfering energy field incident on and between the tip electrode 142 and ring electrode 140 to send a power potential or voltage 133 to the active circuits. In one example, a rectification circuit may be employed as will be appreciated by those skilled in the art. Alternately, however, any process that creates a signal from the interfering energy field may be employed in the present disclosure.

Each active circuit can take one or a variety of actions to combat the interfering incident energy. For example, an active switch, such as a field effect transistor (FET), can open the connection between the ring electrode 140 and the conductor 148 that couples the ring electrode 140 to the IMD 20. Alternately a low-pass filter could be switched into series between the ring electrode 140 and the conductor 148. In one embodiment, a resonant filter (also known as a notch or band-stop filter) could be switched into series between the ring electrode 140 and the conductor 148. Similar actions can be taken by the active circuit 146a for the tip electrode 142.

The active circuit 146c offers the possible action of shunting the ring electrode 140 and the tip electrode 142. The shunting could be a capacitor, a filtering circuit, a resistor or some actively switched combination thereof. For the resistive shunting, it may be desirable to dissipate heat energy from the resistor in a heat absorbing material surrounding the active circuit 146c or via a heat-sink in an energy transferring connection to the resistor. By way of example, and not as a limitation, other actions that may be taken alone or in combination with any of the actions described above are shown below in Table 1.

TABLE 1

Open tip and ring electrodes from conductors via active
circuit 146a & 146b
Shunt tip and ring electrodes via active circuit 146c
Shunt tip and ring electrodes with impedance via active
circuit 146c
Open tip electrode from conductor via active circuit 146a
and shunt conductor with impedance via active circuit 146c
Open tip and ring electrodes from conductors via active
circuit 146a & 146b and short conductors via active circuit
146c

The actions described above may be taken alone or in combination with one another. For example, in response to the power signal received from power circuit 133, active circuits 146a and 146b may open (in the case of a switch) and a shunt with impedance may be placed between ring electrode 140 and tip electrode 142.

Each action, or combination of actions, are designed to reduce or eliminate damaging effects of the incident MRI/EMI energy. Accordingly, the techniques of the present disclosure offers a variety of options to address patient tissue heating issues or damage to circuitry of the IMD. Although FIG. 5 illustrates a configuration with three active circuits 146a, 146b, 146c, active satellite circuit 146 may include more or fewer than three active circuits. For example, active satellite circuit 146 may include active circuits 146a and 146b and not have a shunt active circuit 146c. In another example, active satellite circuit 146 may include only a shunt active circuit 146c and not include active circuits 146a and 146b. In yet other examples, active satellite circuit 146 may include a plurality of series active circuits coupled between tip electrode 142 and IPG 120, a plurality of series active circuits coupled between ring electrode 140 and IPG 120, or a plurality of shunt active circuits coupled between tip electrode 142 and ring electrode 140, or any combination thereof. In a further example, an active circuit may be coupled between tip electrode 142 and a metallic lead sleeve head (not shown) to redirect current from tip electrode 142 to the metallic lead sleeve head. An example metallic lead sleeve head is described in U.S. Patent Application No. 2007/0299490 to Yang et al. entitled, “RADIOFREQUENCY (RF)-SHUNTED SLEEVE HEAD AND USE IN ELECTRICAL STIMULATION LEADS,” which is incorporated by reference herein in its entirety.

The power signal 133 can also constitute a signal to the controller of IPG 120 indicating that an interfering electromagnetic field is present. That is, since the power signal 133 is provided only in the presence of an interfering external field, merely sending the signal to the controller will allow the controller to determine whether to take some action of its own, such as enter a safe mode or altering the therapy delivered to the patient. In one embodiment, the power signal may be communicated to the controller via a separate conductor 147’. In this embodiment, the power signal can then be used by the controller of IPG 120 (122a in FIG. 3) so that the implanted medical device can be protected without using power from the battery (25 in FIG. 3) that is used to generate therapy. Alternately, a communication circuit (“COMM”) 147 can be included in the active circuit satellite 146 so that the power signal can be communicated to the controller via conductor 142′ from the tip electrode to the device or conductor 148 from the ring electrode to the device in any suitable multiplexed manner so as not to interfere with the delivery of therapy. For example, time division multiple access (TDMA) communication signals may be multiplexed with pacing pulses. In other embodiments other communication techniques may be used such as frequency division multiple access (FDMA), code division multiple access (CDMA), or any other channel access technique In another embodiment, multiplexed communication need not be used provided that the communication signals have an amplitude that are sub-threshold to a pacing pulse.

In another embodiment, communication between the active circuit satellite 146 and the satellite communication circuit 122c may be bi-directional. In this case, the satellite communication circuit 122c may send instructions to the active circuit satellite 146 or individual ones of active circuits 146a, 146b, 146c regarding what actions to take with the now powered active circuits. Controller 122a of IPG 120 may, for example, analyze the signal further in response to receiving a power signal or other signal indicating the presence of the interfering electromagnetic field and instruct one or more of active circuits 146a, 146b, 146c regarding the action to take. Controller 122a may, for example, instruct one of the active circuits to switch in a first resonant filter between tip electrode and the IPG 120 for a 1.5 T MRI and switch in a second resonant filter between tip electrode and the IPG 120 for a 3.0 T MRI. Changes in actions taken can be based upon the strength or nature of the interfering field or can be programmed by the physician using the external programmer 50 discussed above in conjunction with FIG. 2. In yet another embodiment, the one or more active circuits 146a, 146b, 146c may communicate with one another to change actions taken in response to the interfering field.

With reference to FIG. 6, another embodiment of the active circuit satellite 146 is shown. In this embodiment, which operates to take actions much as discussed above in conjunction with FIG. 5, a narrow-band MRI/EMI detector (“DET”) 135 is included that within the MRI/EMI power circuit 132 to generate a detect signal 139 when a particular interfering field of interest is detected. The narrow-band MRI/EMI detector 135 can be capable of detecting multi-level field strengths and/or can be capable of discriminating between different field ranges. In one embodiment, the narrow-band MRI/EMI detector 135 includes a respondent circuit tuned to an MRI frequency to detect when the patient is undergoing an MRI procedure. The active circuits 146a, 146b and 146c of this embodiment are coupled to the detect signal 139 and do not take action upon becoming active as was the case in the embodiment of FIG. 5. Rather, once active, the active circuits 146a, 146b and 146c check to see if the detect signal 139 is present prior to taking action. This embodiment offers added patient protection so that an errant (but less threatening) field does not cause an alteration of therapy to the patient. This technique may, for example, result in the active circuits 146 taking action in an MRI field and not taking action in another interfering field, such as an electronic article surveillance (EAS) gate. Instead, actions are only taken after confirmation of the presence of a particular field (or fields) of interest has been detected.

In this embodiment, the detect signal 139 constitutes a signal to the controller of IPG 120 indicating that an interfering electromagnetic field is present. Since the detect signal 139 is provided only in the presence of a particular interfering external field, sending the detect signal 139 to the controller will allow the controller to determine whether to take some action, such as synchronizing pacing, entering a safe mode or altering the therapy delivered to the patient. In one embodiment, the detect signal 139 may be communicated to the controller via a separate conductor 147′. Additionally, the power signal could also be sent via the conductor 147′ so that the power can be used by the controller (122a in FIG. 3) so that the implanted medical device can be protected without using power from the battery (25 in FIG. 3) that is used to generate therapy. Alternately, a communication circuit 147 can be included in the active circuit satellite so that the detect signal can be communicated to the controller in a multiplexed manner so as not to interfere with the delivery of therapy. For example, time division multiple access (TDMA) communication signals may be multiplexed with pacing pulses. In other embodiments other communication techniques may be used such as frequency division multiple access (FDMA), code division multiple access (CDMA), or any other channel access technique In another embodiment, multiplexed communication need not be used provided that the communication signals have an amplitude that are sub-threshold to a pacing pulse.

With reference to FIG. 7a through FIG. 7e, exemplary embodiments of the series active circuits 146a or 146b are shown. It will be appreciated by those skilled in the art that any type of active circuit may be used within the meaning of the present disclosure depending upon the desired protection of the lead designer. FIG. 7a illustrates a basic approach to decoupling the interfering energy from the tip and/or ring electrodes. As shown, a controlled switch 700 is caused to open via the power signal 133 (or the detect signal 139 in the embodiment of FIG. 6). The controlled switch 700 may, for example, comprise an FET, MEMS switch or other switching mechanism.

With reference to FIG. 7b through 7f, various examples of active circuits are shown. In normal operation, controlled switch 700 is closed and controlled switch 702 is open. In response to the power signal 133 (or the detect signal 139 in the embodiment of FIG. 6), controlled switch 700 is opened and controlled switch 702 is closed to place components in series with the tip and/or ring electrode. For example, FIG. 7b series connects a notch filter 704 while FIG. 7c series connects a low-pass filter 706. In FIG. 7d, a phase shifting element 708 may be used in series with the tip and/or ring electrode to phase shift the incident interfering on either the tip or ring electrode energy by 180 degrees. Once phase shifted, the tip and ring electrodes could be shunted (as discussed below in conjunction with FIG. 8a) to cause destructive cancellation of the interfering energy. In FIG. 7e, an active circuit is shown in an exemplary embodiment. As illustrated, a signal processor (e.g., digital signal processor (DSP), microprocessor (μP)) 710 may be switched into series with the tip and/or ring electrode to provide filtering, signal processing or other signal evaluation or information collection. As a final example, FIG. 7f illustrates a series impedance that can be placed in series with the tip or ring electrode. Once in series, the power level of the interfering signal could be reduced to a level to not cause injury to the patient or damage to the implantable medical device. However, the power of the therapy delivered to the patient would also be reduced. Accordingly, the implantable medical device may determine to increase the power level of the therapy to compensate for the series impedance.

With reference to FIG. 8a through FIG. 8e, exemplary embodiments of the shunt active circuit 146c are shown. It will be appreciated by those skilled in the art that any type of active circuit may be used within the meaning of the present disclosure depending upon the desired protection of the lead designer. FIG. 8a illustrates a basic approach to shunting the tip and/or ring electrodes. As shown, a controlled switch 800 is caused to close via the power signal 133 (or the detect signal 139 in the embodiment of FIG. 6) to shunt the tip and ring electrodes. With reference to FIG. 8b through 8e, various examples of active circuits are shown. In these embodiments, controlled switch 800 is closed to shunt components between the tip and ring electrode. For example, FIG. 8b shunts a high-pass filter 804 while FIG. 8c shunts a band-pass filter 806 between the tip electrode 142 and the ring electrode 140. In FIG. 8d, a resistive element 808 may placed in shunt between with the tip electrode 142 and the ring electrode 140 to dissipate heat energy created by current flowing through the resistive element 808. Those skilled in the art will appreciate that it may be beneficial to use a head dissipating structure (e.g., a heat sink) or coating 810 to dissipate any heat generated in a controlled and safe manner. As a final example, FIG. 8e, shows an active circuit including a signal processor (e.g., digital signal processor (DSP), microprocessor (μP)) 812 may be in shunt between the tip electrode 142 and the ring electrode 140 to provide filtering, phase shifting, signal processing or other signal evaluation or information collection.

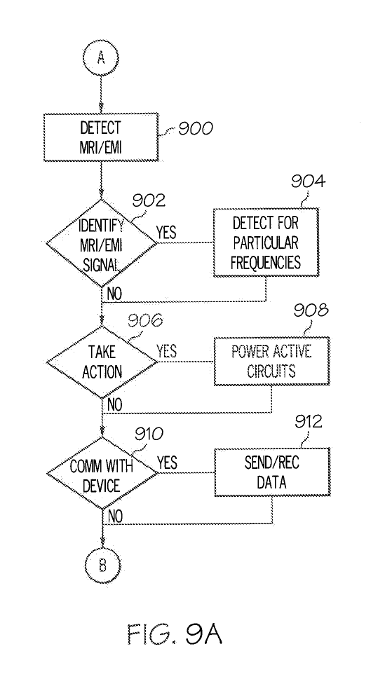

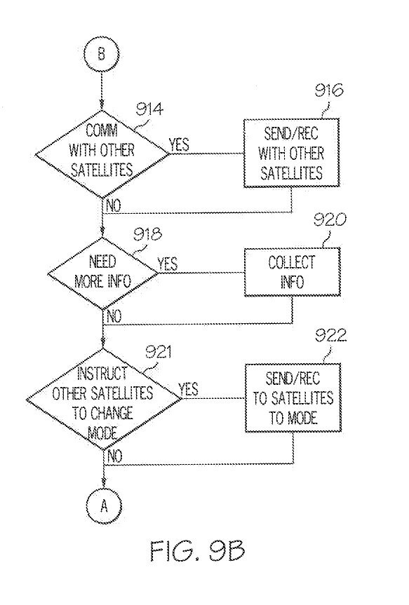

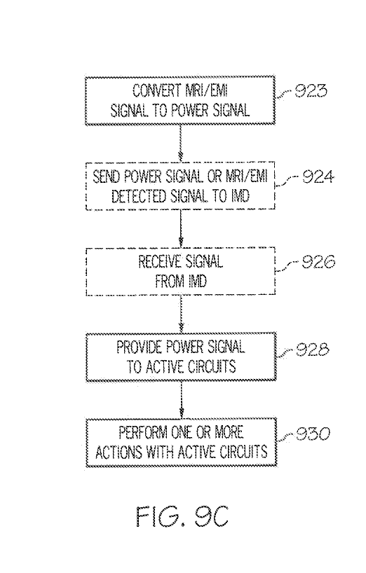

With reference to FIG. 9a and FIG. 9b, exemplary actions that may be taken by the active circuit satellites are shown. At step 900, the MRI/EMI interfering energy is detected such as by the power circuit 133 (FIG. 5) or the detector 135 (FIG. 6). Next, an active circuit satellite may choose to use broadband circuit to analyze or combat the interfering energy. If so, step 904 powers the broadband circuits using power derived from the interfering energy as discussed above. Next, decision 906 may determine whether to use narrowband circuits to combat the interfering energy. If so, then step 908 powers such circuits for the protection of the patient.

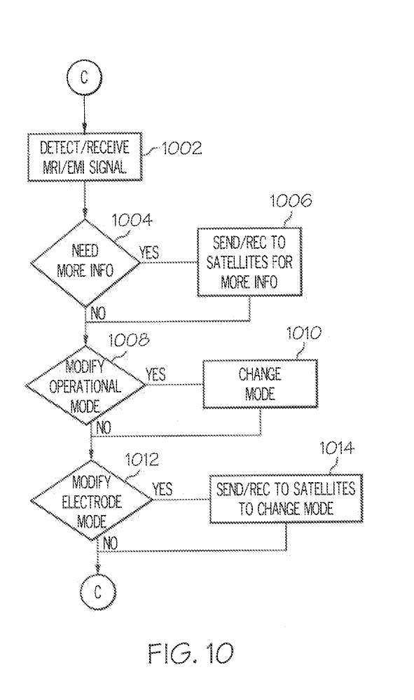

Decision 910 determines whether communication with the implantable medical device is required. If so, step 912 communicates with the implantable medical device as discussed above in conjunction with FIG. 5 and FIG. 6. A basic communication would be to merely signal the presence of the interfering energy so that the implantable medical device can determine to take actions itself as discussed below in conjunction with FIG. 10. Besides communicating with the implantable medical device, an active circuit satellite could determine at 914 (FIG. 9b) whether communication with other active circuit satellites is needed. If so, data is sent and received in step 916 using any form of multiplexed communication desired to be employed by the lead designer. Decision 918 determines whether more information about the interfering signal is needed. If so, step 920 collects such information, which can be provided to the implantable medical device or other active circuit satellites. Finally, decision 921 determines whether one active circuit satellites should instruct other active circuit satellites to change their operational mode. In this way, protective action may be taken independent of the implantable medical device for the benefit of the patient.

With reference to FIG. 9c, exemplary actions that may be taken in step 908 are shown. In step 922, the interfering energy is converted to a power signal. Optionally, Step 924 sends the power signal to the implantable medical device if not already performed by another active circuit satellite. As discussed above, this step allows the controller 122a of the implantable medical device so that the implantable medical device can be protected without drawing upon or depleting the batter 25 used to generate therapy. Step 926 is also an optional step that may be used in some embodiments to receive a signal or instruction from the implantable medical device. In step 928, the active circuits are powered and controlled to take actions (step 930) against damage or injury to the patient that may occur due to the presence of the interfering energy.

With reference to FIG. 10, another embodiment of exemplary actions that may be taken by the implantable medical device or the active circuit satellites is shown. At step 1002, the MRI/EMI interfering energy is detected (by the active circuit satellites) or received (by the implantable medical device). Decision 1004 determines whether more information about the interfering signal is needed. If so, step 1006 informs one or more active circuit satellites to collect such information. This information may be provided back to the implantable medical device and/or other active circuit satellites. Decision 1008 determines whether to modify the operational mode or the implantable medical device or the active circuit satellites. By way of example and not a limitation, the implantable medical device could enter a safe mode, the pacing energy could be increased, the pacing timing could be increased, decreased or made rate responsive. Additionally, or alternatively, the implantable medical device may synchronize the delivery of therapy to some aspect of the interfering signal such as the MRI gradient field timing. In the case of the active circuit satellites, each active circuit satellite could determine to power and control one or more of the active circuits either upon instruction from the implantable medical device or independently. Thus, the goal of decision 1008 is maintain safety for the patient either at the system level or independently at the lead level.

In decision 1012, it is determined whether to modify one or more electrode modes currently in operation by the one or more active circuit satellites. If so, step 1014 sends data to (and may receive from) the active circuit satellites to, for example, change the particular electrode that delivers therapy or senses information from the patient. This can be done by the implantable medical device or by one or more of the active circuit satellites. Those skilled in the art will appreciate that many number of other electrode modes could be entered, changed or suspending depending upon the desire of the lead designer.

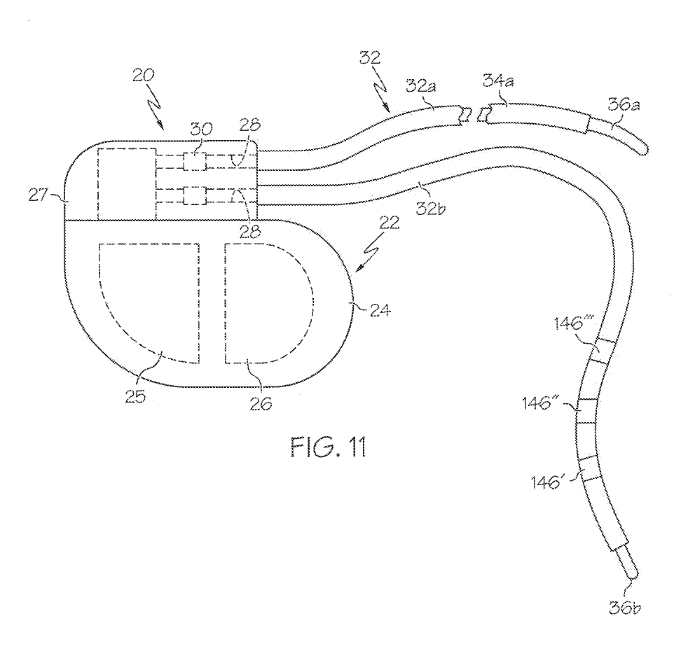

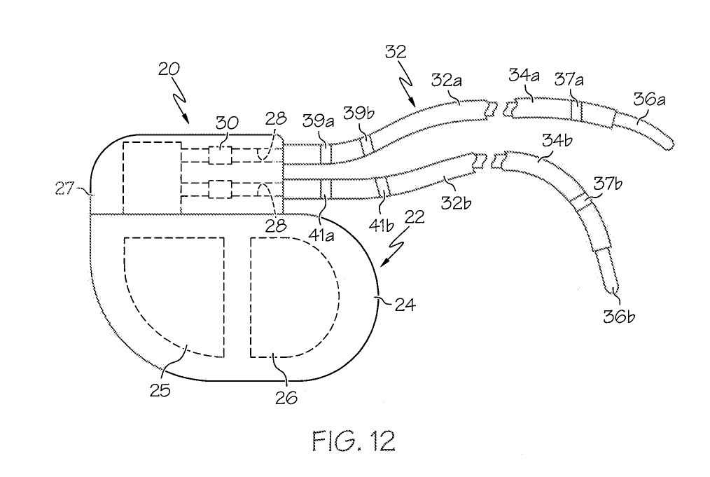

With reference to FIG. 11, another embodiment of the disclosure employs multiple active circuit satellites 146′, 146″ and 146′″ positioned in series along the lead 32b. Multi-electrode leads are particularly advantageous for left-heart applications where the optimum electrode(s) are determined empirically after lead implantation. Each active circuit satellite operates as described above in conjunction with FIG. 5 and FIG. 6 depending upon the configuration chosen. For multi-electrode (satellite) leads, communication from the satellite communication circuit 122c to the active circuit satellites may be implemented using a multiplex (such as TDM) communication protocol so that the diameter of the lead does not increase by the addition of separate communication conductors for each active circuit satellite.

With reference to FIG. 12, another embodiment of the disclosure employs active circuit satellites within or near ring electrodes (39a and 39b of lead 32a and 41a and 41b of lead 32b) to offer protection to the implantable medical device 20 by positioning the active circuit satellites near the proximal end of the leads. However, those skilled in the art will appreciate that the active circuit satellites could be positioned elsewhere along the lead. In one example, interfering energy incident between electrodes 39a and 39b of lead 32a could be converted to power active circuits as discussed in conjunction with FIG. 5 and FIG. 6 or the protection from damage to internal circuitry 26. Generally, electrodes near the proximal end of the lead are not used to deliver therapy. Instead they are used to receive the interfering energy to create power that can be used by the implantable medical device to power active circuits to protect the implantable medical device from damage. Alternately, electrodes to receive interfering energy could be placed in the connector block 27. Also, as discussed above in conjunction with FIG. 5 and FIG. 6 power would be delivered to the implantable medical device by a conductor within the lead so that MRI/EMI protection circuitry can be powered without using energy from the battery 25. In this way, the techniques of the present disclosure may find application along the lead to use the incident interfering field to combat dangerous or harmful effects of the incident interfering field.

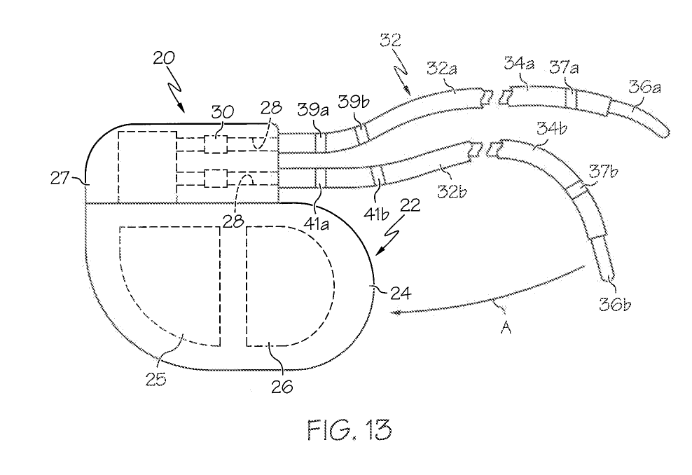

With reference to FIG. 13, another embodiment of the disclosure uses the implantable medical device housing 24 as an electrode so that only one electrode on the lead (for example 36b) is needed to create power from the interfering signal. In other words, the power circuit 132 converts signals incident on and between the electrode and a conductive portion of a housing of the implantable medical device to a power potential for the one or more active circuits. Conduction takes place along path A through the patient's tissue. As discussed above, the power signal is then used to control active circuits to protect the patient and/or implantable medical device.

As discussed above in conjunction with FIG. 3, the MRI/EMI powered active circuit satellite lead 124 includes a proximal end 134, a distal end 136, a body 138, at least one first or ring electrode 140, at least one second or tip electrode assembly 142 and one or more active circuit satellites 146. At the proximal end 134, the distal end 136 and the body 138 of the lead 124 can comprise any suitable proximal end, distal end and body, such as that associated with the CAPSUREFIX™ line of cardiac leads available from Medtronic, Inc. of Minneapolis, Minn., only the modifications or augmentations to the proximal end 134, the distal end 136 and the body 138 of the lead 124 will be discussed in detail herein. The proximal end 134 of the lead 124 can include a connector terminal 30 (FIG. 12) that can electrically couple the lead 124 to the connector body 27 of the IPG 20. Typically, the connector body for implantable medical devices are standardized so that the implantable medical lead of one manufacturer may be used with the implantable medical device of another manufacture. In this way, the implantable medical lead of the present disclosure having one or more active circuit satellites that self-power active circuit therein can render any compatible implantable medical device MRI/EMI safe since, in basic embodiments, no separate power or command is required to have the active circuit become powered and take actions to combat the interfering energy.

As discussed above, the distal end 136 typically terminates within an anatomical structure adjacent to the desired location for the delivery of the therapy to the heart 42, as generally known, and illustrated in FIG. 2. As will be discussed, the ring electrode 140 and the tip electrode 142b can be coupled at or near the distal end 136 to deliver a therapy to an atrium A of the heart 42 (FIG. 2) or to the left or right ventricle. It will be understood, however, that the ring electrode 140 and the tip electrode 142b can be coupled at any desired location along the body 138 of the lead 124. The body 138 of the lead 124 can extend from the proximal end 134 to the distal end 136. The body 138 of the lead 124 can comprise a bifilar coil 148. The bifilar coil 148 can include a pacing transmission member or pacing conductor 150 and at least one or multiple sensing/control transmission members or sensing/control conductors 152.

The pacing conductor 150 and the sensing/control conductor 152 can each be insulated to conduct or carry electrical signals along the body 138 of the lead 124. The pacing conductor 150 can be in communication with the pulse generator 122b to conduct or carry electrical pulses from the IPG 20 to the ring electrode 140 or the tip electrode assembly 142 to pace the heart 42. The ring electrode 140 can be disposed near the distal end 136 of the lead 124 (FIG. 3). The ring electrode 140 can be generally annular, and can include an outer surface 140a and an inner surface 140b, as shown in FIG. 4. The outer surface 140a can be adjacent to the anatomical structure, such as the heart 42. The outer surface 140a of the ring electrode 140 can have a surface area that can range from about 0.5 square millimeters to about 40 square millimeters, but generally need only be large enough to receive the active circuit satellite 146. The outer surface 140a can enable the ring electrode 140 to sense the electrical activity of the heart 42. In this regard, the control system 122 can be in communication with the ring electrode 140 to receive the sensed electrical activity of the heart 42. As known, based on the sensed electrical activity of the heart 42, the controller 122a of the IPG 20 can determine if a therapy is needed for the patient 40.

With reference again to FIG. 4, the tip electrode assembly 142 can also be in communication or electrically coupled with the active circuit satellite to receive the electrical pulses from the IPG 20. The tip electrode assembly 142 can include a transmission coil 142a, a tip electrode 142b and optionally, a fixation mechanism 142c. The fixation mechanism 142c, if employed, can be used to secure the tip electrode assembly 142 to a desired location in the anatomical tissue, such as the heart 42, as illustrated in FIG. 2.

While specific examples have been described in the specification and illustrated in the drawings, it will be understood by those of ordinary skill in the art that various changes may be made and equivalents may be substituted for elements thereof without departing from the scope of the present disclosure as defined in the claims. Furthermore, the mixing and matching of features, elements and/or functions between various examples is expressly contemplated herein so that one of ordinary skill in the art would appreciate from this disclosure that features, elements and/or functions of one example may be incorporated into another example as appropriate, unless described otherwise, above. Moreover, many modifications may be made to adapt a particular situation or material to the teachings of the present disclosure without departing from the essential scope thereof. For example, the device may include an RF generator that generates the RF signal used to power the EMI/MRI protection circuitry. Therefore, it is intended that the present disclosure not be limited to the particular examples illustrated by the drawings and described in the specification as the best mode presently contemplated for carrying out this disclosure, but that the scope of the present disclosure will include any embodiments falling within the foregoing description and the appended claims.

1. An implantable lead for use with an implantable medical device comprising:
a lead body having a proximal end configured to couple to a housing of the implantable medical device and a distal end configured to be located adjacent to an anatomical tissue of a patient;
an electrode positioned near the distal end of the lead;
a conductor extending from the proximal end of the lead to the distal end of the lead and in electrical communication with the electrode;
one or more active circuits within the at least one lead, each active circuit including circuitry for performing an action to reduce negative effects of an interfering electromagnetic field; and
at least one power circuit positioned within the lead that converts the interfering electromagnetic field into power for the one or more active circuits such that the one or more active circuits become operational when the interfering electromagnetic field is present.

2. The implantable lead of claim 1, wherein the interfering electromagnetic field comprises at least one of a magnetic or electric field generated during a magnetic resonance imaging (MRI) scan.

3. The implantable lead of claim 1, wherein the electrode comprises a first electrode and the conductor comprises a first conductor, the implantable medical lead further comprising:
a second electrode positioned near the distal end of the lead;
a second conductor extending from the proximal end of the lead to the distal end of the lead and in electrical communication with the second electrode.

4. The implantable lead of claim 3, wherein the at least one power circuit converts signals incident on and between the first electrode and the second electrode to a power potential for the one or more active circuits.

5. The implantable lead of claim 4, wherein at least one of the active circuits is positioned within and coupled to the second electrode.

6. The implantable lead of claim 3, further comprising:
a third conductor extending from the proximal end of the lead to the distal end of the lead; and
communication circuitry that is electrically coupled to the third conductor to communicate a signal from the at least one power circuit to the implantable medical device to indicate that the interfering electromagnetic field is present.

7. The implantable lead of claim 1, further comprising communication circuitry for communicating a signal via the at least one conductor to indicate that the interfering electromagnetic field is present.

8. The implantable lead of claim 1, wherein at least one of active circuits receives instructions from the implantable medical device identifying an action to be taken to reduce negative effects of the interfering electromagnetic field.

9. The implantable lead of claim 1, further comprising a circuit to detect the presence of one or more frequencies of interest, wherein the one or more active circuits perform the action in response to detecting the presence of at least one of the frequencies of interest.

10. The implantable lead of claim 1, wherein the at least one power circuit converts signals incident on and between the electrode and a conductive portion of a housing of the implantable medical device to a power potential for the one or more active circuits.

11. The implantable lead of claim 1, further comprising a heat dissipating structure to reduce heating within the one or more active circuits.

12. The implantable lead of claim 1, which includes a standardized coupler for connection to various implantable medical devices, wherein the one or more active circuits for reducing or eliminating negative effects of an interfering electromagnetic field of the implantable medical lead causes any of the implantable medical devices coupled to the implantable medical lead to be MRI safe.

13. A method comprising:
converting an interfering electromagnetic field to a power signal;
powering at least one active circuit positioned near a distal end of an implantable medical lead with the power signal; and
performing an action with the at least one active circuit after receiving the power signal to reduce negative effects of the interfering electromagnetic field.

14. The method of claim 13, further comprising:
detecting the presence of a particular frequency of interest within the interfering electromagnetic field; and
performing the action with the at least one active circuit upon detecting the presence of the particular frequency of interest.

15. The method of claim 13, further comprising communicating a signal to the implantable medical device to which the lead is coupled to indicate the presence of the interfering electromagnetic field.

16. The method of claim 15, further comprising performing an action with the implantable medical device in response to communicated signal.

17. The method of claim 16, wherein performing the action with the implantable medical device includes modifying timing for delivery of a therapy.

18. The method of claim 16, wherein performing the action with the implantable medical device includes modifying delivered energy of a therapy.

19. The method of claim 16, wherein performing the action with the implantable medical device includes configuring the implantable medical device into a safe mode of operation.

20. The method of claim 16, wherein performing the action with the implantable medical device includes synchronizing delivery of therapy to a parameter of the interfering electromagnetic field.

21. The method of claim 15, wherein communicating the signal to the implantable medical device comprises communicating the signal to the implantable medical device over a conductor via which therapy is delivered.

22. The method of claim 13, further comprising receiving information from an implantable medical device to which the lead is coupled that indicates the action that the at least one active circuit performs in the presence of the interfering electromagnetic field.

23. The method of claim 13, wherein:
converting the interfering electromagnetic field to the power signal comprises converting a signal generated during a magnetic resonance imaging (MRI) scan to the power signal; and
performing the action with the at least one active circuit comprises performing the action with the at least one active circuit to reduce negative effects of the signal generated during the MRI scan.

24. An implantable medical device operable to provide therapy to anatomical tissue of a patient, comprising:
a housing having a controller including circuitry for generating the therapy;
a lead having a proximal end coupled to the housing and a distal end located adjacent to an anatomical tissue of a patient, the lead further comprising:
an electrode positioned near the distal end of the lead;
a conductor extending from the proximal end of the lead to the distal end of the lead and in electrical communication with the electrode; and
at least one active circuit satellite positioned within the lead and near the electrode and having a power circuit for converting an interfering electromagnetic field into power for controlling one or more active circuits within the at least one active circuit satellite, each active circuit including circuitry for performing an action to reduce negative effects of the interfering electromagnetic field.

25. The implantable lead of claim 24, wherein the electrode comprises a first electrode and the conductor comprises a first conductor, the implantable medical lead further comprising:
a second electrode positioned near the distal end of the lead;
a second conductor extending from the proximal end of the lead to the distal end of the lead and in electrical communication with the second electrode.

26. The implantable lead of claim 25, wherein the at least one power circuit converts signals incident on and between the first electrode and the second electrode to a power potential for the one or more active circuits.

27. The implantable medical device of claim 24, wherein the interfering electromagnetic field comprises a magnetic or electric field generated during a magnetic resonance imaging (MRI) scan.

28. The implantable medical device of claim 24, which includes communication circuitry for communicating the signal via the conductor for communicating a signal via the conductor to indicate that the interfering electromagnetic field is present.

29. The implantable medical device of claim 24, which includes communication circuitry for communicating the signal via the conductor to deliver the therapy in a manner synchronized to the delivery of the therapy so as not to interfere with the delivery of the therapy.

30. The implantable medical device of claim 26, wherein each of the active circuit satellites include a predetermined address so as to be separately addressable for communication to or from the controller.

31. The implantable medical device of claim 24, wherein the active circuits receives instructions from the implantable medical device identifying an action to be taken to reduce negative effects of the interfering electromagnetic field.

32. The implantable lead of claim 24, further comprising a circuit to detect the presence of one or more frequencies of interest, wherein the one or more active circuits perform the action in response to detecting the presence of at least one of the frequencies of interest.
